(19) United States
(12) Patent Application Publication
(10) Pub. No.: US 2011/0097702 A1
(43) Pub. Date: Apr. 28, 2011

(54) Methods and compositions for in situ detection of microorganisms on a surface

(76) Inventor: Kent J. Voorhees, Golden, CO (US)

(21) Appl. No.: 11/395,485

(22) Filed: Mar. 30, 2006

Related U.S. Application Data
(60) Provisional application No. 60/667,291, filed on Mar. 31, 2005.

(51) Int. Cl. C12Q 1/70

(52) U.S. Cl. 435/5

(57) ABSTRACT

Compositions and methods for in situ detection of one or more target microorganisms on a surface and preferably on a hard surface. Compositions and methods of the invention are based on the specificity of certain bacteriophage for target microorganisms. Bacteriophage are modified to express detectable biomarkers in the presence of the target microorganism, the detectable markers being detectable on the surface being tested using a portable detection device. Only living microorganisms are detected using the methods and compositions of the invention.

### CROSS-REFERENCE TO RELATED APPLICATIONS

This application claims priority to and incorporates by reference U.S. Provisional Patent Application No. 60/667,291, filed Mar. 31, 2005.

The application is related to U.S. patent application Ser. No. 10/823,294, filed Apr. 12, 2004 and U.S. patent application Ser. No. 10/249,452, filed Apr. 10, 2003, each of which is incorporated by reference in its entirety.

### STATEMENT REGARDING FEDERALLY SPONSORED RESEARCH

The invention was made with United States Government support under Project No. 4-42209 awarded by the Armed Forces Institute for Pathology. The United States Government has certain rights in the invention.

### BACKGROUND OF THE INVENTION

a. Field of the Invention

The invention generally relates to the field of microorganism detection, and more particularly to in situ detection of microorganisms on surfaces.

b. Statement of the Problem

Detection of microorganisms on hard surfaces, for example detection of pathogenic bacteria on hard surfaces in a building, is a major issue with significant economic and social implications. For example, after the events in the United States of America on Sep. 11, 2001, several domestic environments were closed due to both real and imagined anthrax contamination. The amount of time and money consumed during the clean-up of those environments was considerable, in some cases taking several months to process a site. Presently, there are few, if any, protocols for dealing with potential microorganism contamination (intentional or unintentional) in a domestic environment beyond the sampling of various surfaces and using standard laboratory based microbiological techniques for detection and/or identification of potential pathogens in the sample. This is especially true for the detection of viable microorganisms on surfaces.

Random surface sampling of an environment is typically performed at various sites within the environment and the samples are then processed using standard laboratory microbiological methods. The most prevalent of these laboratory methods for microorganism detection relies on substrate-based assays to test for the presence of specific bacterial pathogens. See Robert H. Bordner, John A. Winter, and Pasquale Scarpino, Microbiological Methods For Monitoring The Environment, EPA Report No. EPA-600/8-78-017, U.S. Environmental Protection Agency, Cincinnati, Ohio, 45268, December 1978. These techniques are generally easy to perform, do not require expensive supplies or laboratory facilities, and offer high levels of selectivity. However, these methods are hindered by the requirement to first grow or cultivate pure cultures of the targeted organism which can take twenty-four hours or longer. This time constraint severely limits the effectiveness to provide rapid response to the presence of virulent strains of microorganisms within the tested environment.

Alternative methods for laboratory testing of samples taken from a surface include methods based on molecular biology techniques. These techniques are quickly gaining acceptance as valuable alternatives to standard microbiological tests. Serological methods have been widely employed to evaluate a host of matrices for targeted microorganisms. See David T. Kingsbury and Stanley Falkow, Rapid Detection And Identification of Infectious Agents, Academic Press, Inc., New York, 1985 and G. M. Wyatt, H. A. Lee, and M. R. A. Morgan, Chapman & Hall, New York, 1992. These tests focus on using antibodies to first trap and then separate targeted organisms from other constituents in complicated biological mixtures. Once isolated, the captured organism can be concentrated and detected by a variety of different techniques that do not require cultivating the biological analyte. One such approach, termed “immunomagnetic separation” (IMS), involves immobilizing antibodies to spherical, micro-sized magnetic or paramagnetic beads and using these beads to trap targeted microorganisms from liquid media. The beads are easily manipulated under the influence of a magnetic field facilitating the retrieval and concentration of targeted organisms. Moreover, the small size and shape of the beads allow them to become evenly dispersed in the sample, accelerating the rate of interaction between bead and target. These favorable characteristics lead to reductions in assay time and help streamline the analytical procedure, making it more applicable for higher sample throughput and automation.

Downstream detection methods previously used with IMS include ELISA (Kofitsyo S. Cudjoe, Therese Hagtvedt, and Richard Dainty, “Immunomagnetic Separation of Salmonella From Foods And Their Detection Using Immunomagnetic Particle”, International Journal of Food Microbiology, 27 (1995), pp. 11-25), dot blot assay (Eystein Skjerve, Liv Marit Rorvik, and Orjan Olsvick, “Detection Of Listeria Monocytogenes In Foods By Immunomagnetic Separation”, Applied and Environmental Microbiology, November 1990, pp. 3478-3481), electrochemiluminescence (Hao Yu and John G. Bruno, Immunomagnetic-Electrochemiluminescent Detection Of Escherichia coli 0157 and Salmonella typhimurium In Foods and Environmental Water Samples”, Applied and Environmental Microbiology, February 1996, pp. 587-592), and flow cytometry (Barry H. Pyle, Susan C. Broadway, and Gordon A. McFeters, “Sensitive Detection of Escherichia coli 0157:H7 In Food and Water By Immunomagnetic Separation And Solid-Phase Laser Cytometry”, Applied and Environmental Microbiology, May 1999, pp. 1966-1972). Although these tests provide satisfactory results, they are laborious to perform and give binary responses (yes/no) that are highly susceptible to false-positive results due to cross-reactivity with non-target analytes. Another method for identifying whole cellular microorganisms uses IMS coupled to matrix-assisted laser desorption/ionization (MALDI) time-of-flight (TOF) mass spectrometry (MS) (Holland et al., 1996; van Barr, 2000; Madonna et al., 2000).

All of these laboratory approaches offer faster results than do traditional microbiology methods. However, they do not achieve the sensitivity levels that substrate-based assays do, are more expensive, and typically require more highly trained technicians than do classical substrate-based methods.

In addition, other molecular biology techniques are receiving a greater deal of attention in laboratory detection of pathogens in a sample. Polymerase Chain Reaction (PCR) detection of specific microorganisms in a sample involves extraction of the genetic material (RNA and/or DNA) in a sample, amplification of a target genetic sequence specific to the microorganism of interest, and then detection of the amplified genetic material. PCR techniques offer high selectivity owing to the uniqueness of the detected genetic material, high sensitivity because of the substantial amplification of the target genetic material, and rapid results owing to the potentially fast amplification process. However, PCR instruments and reagents are quite expensive and highly trained technicians are needed to perform the tests. In addition, PCR detection is not limited to living microorganisms, but rather will signal the presence of an organism as long as the organism's genetic material is present, i.e., the organism could be present but dead, not posing a threat to the environment.

Some attempts have been made to improve upon substrate-based and molecular biologic-based bacterial detection methods in a sample using bacteriophage infection and/or amplification. Bacteriophages are viruses that have evolved in nature to use bacteria as a means of replicating themselves. A bacteriophage (or phage) does this by attaching itself to a bacterium and injecting its genetic material into that bacterium, inducing it to replicate the phage from tens to thousands of times. Some bacteriophage, called lytic bacteriophage, rupture the host bacterium releasing the progeny phage into the environment to seek out other bacteria. The total incubation time for phage infection of a bacterium, phage multiplication (amplification) in the bacterium, and release of the progeny phage after lysis can take as little as an hour depending on the phage, the bacterium, and the environmental conditions. Microbiologists have isolated and characterized over 5,000 phage species, including many that specifically target bacteria at the species or even the strain level. U.S. Pat. No. 5,985,596 issued Nov. 16, 1999 and U.S. Pat. No. 6,461,833 B1 issued Oct. 8, 2002 both to Stuart Mark Wilson describe such a phage-based assay method. It comprises a lytic phage infection of a sample that may contain bacteria of interest. This is followed by removal of free phage from the sample, target bacteria lysis, and then infection of a second bacterium by the progeny phage where the second bacterium has a shorter doubling time than does the target bacterium. The prepared sample is grown on a substrate and the formation of plaques indicates the presence of the target bacterium in the original sample. This method can shorten the assay time of a traditional substrate-based assay, though assays still take many hours or days because of the requisite culture incubation times. Another problem with these patented method is that it can only be applied to detect bacterium for which a non-specific phage exists that also infects a more rapidly doubling bacterium than the target bacterium. Usage of a nonspecific phage opens the possibility of cross-reactivity to at least the second bacterium in test samples. Thus, this phage-based, plaque assay method is not rapid, can only be applied if a suitable non-specific phage is available, is prone to cross-reactivity problems, and must be performed in a laboratory setting.

Other bacterial pathogen detection methods have abandoned the substrate-based, plaque detection methodology altogether. Many of these methods utilize bacteriophage that have been genetically modified with a lux gene which is only expressed if a target bacterium is present in a sample and is then infected by the modified phage. U.S. Pat. No. 4,861,709 issued Aug. 29, 1989 to Ulitzur et al. is a typical example. A phage that specifically infects a target pathogen is modified to include a lux gene. When the modified phage is added to a sample containing the target bacterium, the phage infects the bacterium, luciferase is produced in the bacterium, and light is emitted. U.S. Pat. No. 5,824,468 issued Oct. 20, 1998 to Scherer et al. describes a similar method. In addition to luciferase-producing gene markers, Scherer et al. describes gene markers that are expressed as detectable proteins or nucleic acids. U.S. Pat. No. 5,656,424 issued Aug. 12, 1997 to Jurgensen et al. describes a method utilizing luciferase (or .beta.-galactosidase) reporter phage to detect mycobacteria. It further describes testing for antibiotic susceptibility. U.S. Pat. No. 6,300,061 B1 issued Oct. 9, 2001 to Jacobs, Jr. et al. describes yet another method for detecting mycobacteria using genetically modified phage, which produces one of several reporter molecules after bacterial infection, including luciferase. U.S. Pat. No. 6,555,312 B1 issued Apr. 29, 2003 to Hiroshi Nakayama describes a method utilizing a gene that produces a fluorescent protein marker rather than a luminescent one. All of these methods take implicit advantage of phage amplification within infected bacteria. For each target bacterium infected in a sample, the marker gene is expressed many times over as the progeny phage are produced. U.S. Pat. No. 6,544,729 B2 issued Apr. 8, 2003 to Sayler et al. adds an additional amplification process. A phage's DNA is modified to include a lux gene. A bioreporter cell is also modified to include a lux gene. The genetically modified phage and bioreporter cells are added to a sample. If the phage infects target bacteria, the target bacteria are induced to produce not only luciferase but also acyl en homoserine lactone N-(3-oxohexanoyl) homoserine lactone (AHL). AHL finds its way into the bioreporter cells, stimulating the production of additional light and additional AHL, which in turn finds its way into additional bioreporter cells resulting in the production of even more light. Thus, an amplified light signal is triggered by the phage infection of the target bacteria. In principle, all of these methods utilizing genetically modified phage make possible: 1) high selectivity because they utilize selectively infecting phage; 2) high sensitivity because the marker gene products can be detected at low levels; and 3) results that are faster than substrate-based methods because the signal can be detected within one or two phage infection cycles. They have two significant drawbacks. First, they are expensive and difficult to implement because suitable phage must be genetically modified for each pathogen to be tested. Second, they often require an instrument to detect the marker signal (light), driving up the cost of tests utilizing genetically modified phage.

U.S. Pat. No. 5,888,725 issued Mar. 30, 1999 to Michael F. Sanders describes a method utilizing unmodified, highly specific lytic phages to infect target bacteria in a sample. Phage-induced lysis releases certain nucleotides from the bacterial cell such as ATP that can be detected using known techniques. Detecting increased nucleotide concentrations in a sample after phage infection indicates the presence of target bacteria in the sample. U.S. Pat. No. 6,436,661 B1 issued Aug. 20, 2002 to Adams et al. describes a method whereby a phage is used to infect and lyse a target bacterium in a sample releasing intracellular enzymes, which react in turn with an immobilized enzyme substrate, thereby producing a detectable signal. While these methods have the advantage of using unmodified phage, they do not derive any benefit from phage amplification. The concentration of detected markers (nucleotides or enzymes) is directly proportional.

U.S. Pat. No. 5,498,525 issued Mar. 12, 1996 to Rees et al. describes a pathogen detection method using unmodified phage and phage amplification to boost the detectable signal. The method calls for adding a high concentration of a lytic phage to a sample. The sample is incubated long enough to allow the phage to infect the target bacteria in the sample. Before lysis occurs, the sample is treated to remove, destroy, or otherwise inactivate the free phage in the sample without affecting the progeny phage being replicated within infected bacteria. If necessary, the sample is subsequently treated to neutralize the effects of any anti-viral agent previously added to the sample. The progeny phage released by lysis are detected using a direct assay of the progeny phage or by using a genetically modified bioreporter bacterium to generate a signal indicating the presence of progeny phage in the sample. In either case, the measured signal is proportional to the number of progeny phage rather than the number of target bacteria in the original sample and, thus, is enhanced as a result of phage amplification. A key disadvantage of this method is that it requires free phage in the treated sample to be destroyed, removed, or inactivated followed by reversal of the virucidal conditions such that progeny phage will remain viable after lysis. These additional processes complicate assays utilizing the method and make them more expensive.

Note that in each of these detection methods, the target surface must first be sampled and then various laboratory procedures performed to determine if a microorganism of interest is present. These laboratory based methods therefore are incomplete, relying on the sampling of the hard surface to obtain the target microorganism prior to a laboratory testing step. In such cases where one or more hard surface sample is positive for a microorganism, a total environment clean-up would likely be necessitated. In cases where no samples were found to include a target microorganism, an uncertainty would remain as to the potential that other non-sampled surfaces within the target area had target microorganisms present but not sampled. Further, a number of the above described detection techniques do not differentiate between alive or dead microorganisms.

What is needed in the art is a detection method combining the sensitivity, simplicity, and/or low cost of substrate-based assays with the rapid results offered by molecular biology diagnostic tests. In addition, what is needed in the art is a detection method that in situ detects living microorganisms over significant areas of surface within a target environment and avoids the problems of having to rely on random sampling to locate a target microorganism.

### BRIEF SUMMARY OF THE INVENTION

The invention provides compositions for in situ detection of target microorganisms on a surface. Compositions of the invention typically include a non-volatile detection composition having a growth media impregnated with one or more bacteriophage specific for target microorganisms. In typical embodiments the bacteriophage are genetically engineered to express a protein that can be detected in the absence of laboratory sampling procedures, for example a fluorophore. Expression of the protein is only accomplished when living target microorganisms are present and able to support the infection of the specific genetically engineered microorganism. In some embodiments the non-volatile growth media is a water and glycerol combination, a water and polyethylene glycol combination or a growth agar for the target microorganism.

The invention also provides in situ methods for detecting target microorganisms on a surface, typically within a domestic environment, for example, a hard surface in rooms of a building, in car interiors, or on objects of interest, e.g., lamps, phones, copy machines, etc. In one preferred embodiment, parent phage are genetically engineered to express detectable marker proteins, for example a fluorophore, and applied to a surface in a non-volatile media. Presence of target microorganism is detected after an appropriate amount of time for the parent phage to absorb to the surface of target microorganism, inject the genetically engineered DNA, and express the detectable the marker protein. Note that additional amplification of progeny phage occurs during this time, leading to additional infection of microorganisms and a repeat of the cycle. Detection of microorganisms is direct, only requiring a device for observing the detection marker (a portable detector specific for the detection marker). In addition, in situ detection methods of the invention are specific for viable microorganisms as expression of the marker protein requires viable target microorganisms for phage DNA amplification and expression, i.e., expression of the detection marker. Embodiments of the invention include modification of the environment, i.e., temperature, growth media, etc, to optimize phage amplification within the target microorganism.

The features, utilities and advantages of the various embodiments of the invention will be apparent from the following more particular description of embodiments of the invention as illustrated in the accompanying drawings.

### BRIEF DESCRIPTION OF THE DRAWINGS

FIG. 1 is an illustration of a bacteriophage;

FIG. 2 illustrates a phage amplification process; and

FIG. 3 is a flow diagram illustrating a method for in situ detection of living microorganisms on a hard surface.

### DETAILED DESCRIPTION OF THE PREFERRED EMBODIMENT

The present invention provides compositions and methods useful in the in situ detection of living microorganisms on a surface, and preferably on a hard surface. In embodiments of the invention, specificity of bacteriophage for a target host organism or microorganism is utilized to determine the presence of the target microorganism. The invention does not require sampling procedures on the surfaces or laboratory-based detection procedures, rather the invention relies on in situ detection of the target microorganism and avoids the inadequacies of sampling and off site detection. In addition, embodiments of the present invention are based on target microorganisms being capable of supporting a bacteriophage infection (i.e., are alive), thereby avoiding positive results where the microorganism in reality does not supply a threat to the user of the surface, i.e., the microorganism is dead.

Generally, embodiments of the invention include a preparation of a detection composition for application to a surface in need of testing. The detection composition is typically composed of a non-volatile media impregnated with a modified bacteriophage. The modified bacteriophage has a known specificity for target microorganism(s) and is capable of expressing a detection marker upon infection of the target microorganism. Presence of the target microorganism on a surface can be determined in situ when the non-volatile media is contacted to the surface and presence of the microorganism is detected through expression of the detection marker by the modified bacteriophage. The media composition provides an environment for the modified bacteriophage to interact with target microorganism on the surface, given a period of time necessary for adequate levels of detection marker to be produced. Typically, a portable detection device is utilized to scan the treated surfaces for identification of marker expression and thereby identification of target microorganism.

### Growth Media

Aspects of the invention provide growth media impregnated with at least one strain of modified or genetically engineered bacteriophage specific for different target microorganisms. The combination of growth media and bacteriophage is referred to herein as a detection composition.

Detection compositions useful with aspects of the invention generally have low coefficients of evaporation, i.e., are non-volatile, so as to facilitate genetically engineered phage absorption to target microorganisms and subsequent phage amplification and expression. The non-volatility of the media allows the media to remain on the surface for an appropriate length of time to allow for phage amplification and microorganism detectionprior to media evaporation. Illustrative growth media for use on surfaces of the invention include non-volatile liquid combinations like water/glycerol or water/polyethylene glycol mixtures. Alternative media compositions are based on growth agar for the target microorganism, for example L-broth. Agar based compositions can include glycerol or other ingredients to minimize agar evaporation. Various amounts of glycerol, for example, can be included in the media, for example a detection composition can include a growth media of 10% glycerol in an aqueous medium.

In an alternative embodiment of the growth media, the evaporation coefficient of the growth media is unimportant, rather the media is any liquid capable of delivering the bacteriophage to the surfaces being tested for microorganism presence and that will not interfere with bacteriophage/microorganism absorption and amplification. In one such embodiment the growth media is simply water or other like aqueous media. A polymer film is layered onto the impregnated aqueous media to encapsulate the media onto the surface being tested, i.e., the impregnated aqueous media is applied to the surface and the polymer film is layered onto the media. The encapsulated media is protected from the surface environment and able to resist evaporation for a sufficient amount of time to allow bacteriophage absorption and amplification in contacted target microorganisms. The polymer films are selected to not interfere with bacteriophage infection or amplification of target microorganisms.

### Bacteriophage:

Methods of the invention rely on the usage of bacteriophage, or simply phage, to detect the presence of target microscopic living organism (microorganism), such as a bacterium, on a surface. In this disclosure “surface” refers to any exposed area that can be viewed with the eye or with a remote camera or other like device and accessible to a detection composition. Preferred embodiments of the invention are directed to in situ detection of microorganisms on “hard surfaces,” which refers to any substantially non-absorbent material, for example, woods, metals, plastics, glass, coated fabrics, and the like, that are accessible to the growth media of the invention.

For purposes of the invention the terms “bacteriophage” and “phage” include bacteriophage, phage, mycobacteriophage (such as for TB and paraTB), mycophage (such as for fungi), mycoplasma phage or mycoplasmal phage, and any other term that refers to a virus that can invade living bacteria, fungi, mycoplasmas, protozoa, and other microscopic living organisms and uses them to replicate itself. Here, “microscopic” means that the largest dimension is typically one millimeter or less. Bacteriophage are viruses that have evolved in nature to use bacteria as a means of replicating themselves. A phage does this by attaching itself to a bacterium and injecting its DNA into that bacterium, inducing it to replicate the phage hundreds or even thousands of times. Some bacteriophage, called lytic bacteriophage, rupture the host bacterium, releasing the progeny phage into the environment to seek out other bacteria. The total incubation time for phage infection of a bacterium, phage multiplication or amplification in the bacterium, to lysing of the bacterium takes anywhere from tens of minutes to hours, depending on the phage and bacterium in question and the environmental conditions.

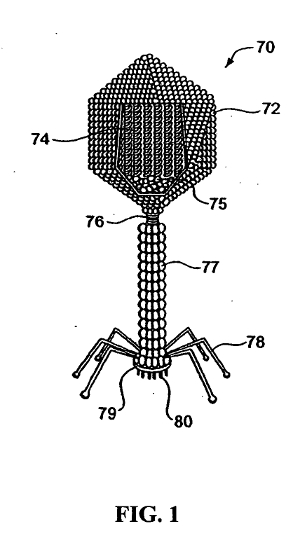

The method taught herein relies on the usage of bacteriophage to detect the presence of one or more target microorganisms, i.e., bacterium, in a sample. A typical bacteriophage 70, in this case MS2-E. Coli is shown in FIG. 1. Structurally, a bacteriophage 70 comprises a protein shell or capsid 72, sometimes referred to as a head, that encapsulates the viral nucleic acids 74, i.e., the DNA and/or RNA. A bacteriophage may also include internal proteins 75, a neck 76, a tail sheath 77, tail fibers 78, an end plate 79, and pins 80. The capsid 72 is constructed from repeating copies of one or more proteins.

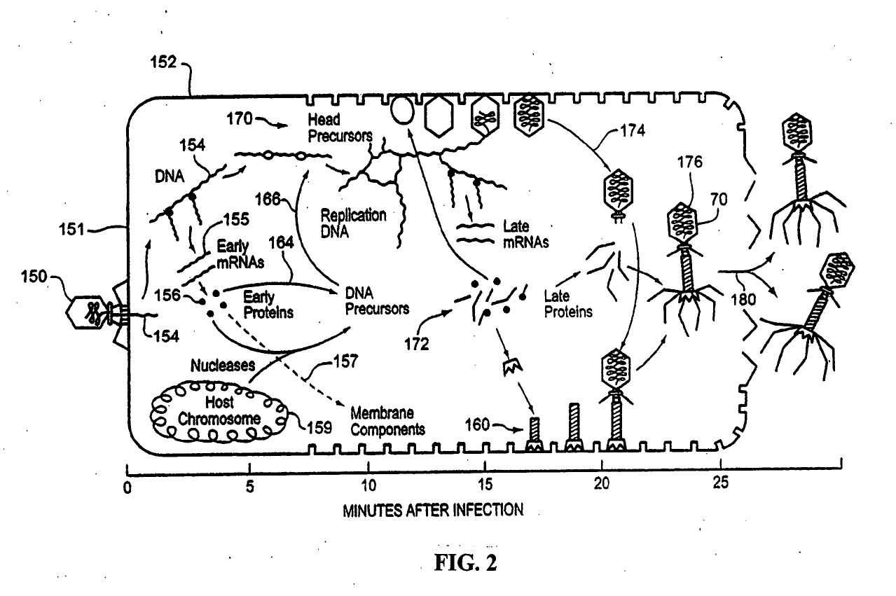

Referring to FIG. 2, when a phage 150 infects a bacterium 152, it attaches itself to a particular site on the bacterial wall or membrane 151 and injects its nucleic acid 154 into that bacterium, inducing it to replicate the phage from tens to thousands of copies. The process is shown in schematic in FIG. 2. The DNA evolves to early mRNAs 155 and early proteins 156, some of which become membrane components along line 157 and others of which utilize bacteria nucleases from host chromosomes 159 to become DNA precursors along line 164. Others migrate along the direction 170 to become head precursors that incorporate the DNA along line 166. The membrane components evolve along the path 160 to form the sheath, end plate, and pins. Other proteins evolve along path 172 to form the tail fibers. When formed, the head releases from the membrane 151 and joins the tail sheath along path 174, and then the tail sheath and head join the tail fibers at 176 to form the bacteriophage 70. Some bacteriophage, called lytic bacteriophage, rupture the host bacterium, shown at 180, releasing the progeny phage into the environment to seek out other bacteria. Lytic phages are typically used in the method disclosed herein. However, non-lytic phages can be used, particularly if they or the bacteria can be activated to release progeny phage or portions of progeny phage after the progeny phage infect the host bacteria.

The total cycle time for phage infection of, for example, a bacterium, phage multiplication or amplification in the bacterium, to lysing of the bacterium takes anywhere from minutes to hours, depending on the phage and bacterium in question and the environmental conditions. As an example, the MS2 bacteriophage infects strains of Escherichia coli and is able to produce 10,000 copies to 20,000 copies of itself within 40 minutes after attachment to the target cell. The capsid of the MS2 phage comprises 180 copies of an identical protein. This means that for each E. coli infected by MS2, upwards of 1.8×10⁶ individual capsid proteins are produced. The process of phage infection whereby a large number of phage and an even larger number of capsid proteins are produced for each infection event is called phage amplification.

Microbiologists have isolated and characterized many thousands of phage species, including specific phages for most human bacterial pathogens. Individual bacteriophage species exist that infect bacterial families, individual species, or even specific strains. Table 1 lists some such phages and the bacterium they infect.

TABLE 1

| PHAGE | BACTERIAL TARGET |
|---|---|
| MS2 | E. coli, Enterococci |
| φA1122 | Yersinia pestis |
| 7 | |
| φFelix 0-1 | Salmonella spp. |
| Chp1 | Chlamydia trachomatis |
| Gamma | B. anthracis |
| A511 | listeria spp |

The present invention takes advantage of these bacteriophage characteristics, such as highly specific bacterial infection, phage amplification to enhance signal, and short incubation/amplification time. In addition, because bacteriophage infect and amplify directly in a microorganism, the present invention is able to be performed in situ on the surface of interest, not requiring a reaction solution or other external materials.

The phage itself may be added to the growth media in a variety of forms to provide the detection composition. The phage may be added in a dry state. The phage may be mixed or suspended directly into the growth media. The phage may be suspended in a vial to which the growth media is added. It also may take any other suitable form.

Embodiments of the invention rely on combining the built-in selectivity characteristics of various bacteriophage with genetic modifications to the bacteriophage to enhance a desirable detection property. Typically, the detection property enhances in situ detection of the amplified phage. For purposes of the present invention “genetically modified” or “genetically engineered” bacteriophage includes bacteriophage in which the DNA is modified, manipulated, or added to in some manner. Typically, the phage of the invention are genetically modified to enhance one or more desirable detection properties.

In one aspect of the invention, phage are genetically engineered to insert DNA into target microorganism that, when encoded within the microorganism, express a detectable marker indicative of phage activity. For example, parent phage can be engineered to incorporate, and thereby inject into host bacteria, DNA that encodes a detectable marker, for example luciferase (see U.S. Pat. No. 4,861,709 incorporated herein by reference, also see sedqley et al., “Real-Time imaging and quantification of bioluminescent bacteria in root canals in vitro,” J. Endod., December (2004) 30(12):894-8, incorporated herein by reference for use of bioluminescence imaging and luminometry), a fluorescent protein marker (see U.S. Pat. No. 6,555,312 B1 incorporated herein by reference) or other like molecules. Presence of the target microorganism is indirectly detected via presence of the expressed detectable marker.

In one embodiment, an amount of genetically modified phage, preferably below a detection limit, is contacted on a surface having a host microorganisms, allowed to infect and incubate, to generate phage progeny, which can be detected via detection of the over-expressed marker. The parent phage is genetically modified to over-express a detectable biomarker, this biomarker is detected in a fast and sensitive manner. This detection process eliminates the need to sample and laboratory test for the presence of the bacteriophage infection and allows the phage infection to be identified in situ on the surface of interest. In addition, the target microorganism must be alive to support infection and ultimately expression of the bacteriophage and the bacteriophage's detectable biomarker.

Expression of marker is expected in from about six to ten hours, and preferably about eight hours. Note, however, that lower concentrations of phage can be used in the media, e.g., 10³-10⁴, but longer incubation periods would be required before adequate or detectable expression of marker would be anticipated. Therefore, flexibility is required in determining the concentration of phage required for in situ testing, factors to consider include: how much phage is available for use in the test, the size of the area to be tested, the time allotted for testing, and the anticipated level or microorganism contamination with the area of testing.

Generally from about 10⁴ to about 10⁶, and preferably about 10⁵ modified phage/ml of media, are required for adequate or detectable expression of marker when exposed to target microorganisms.

### Methods of the Invention:

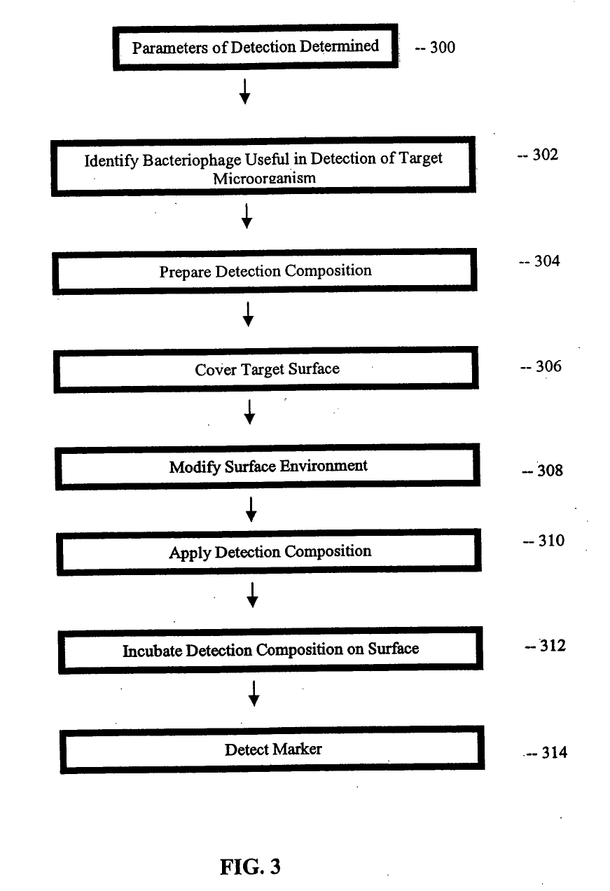

FIG. 3 illustrates a flow chart of the in situ method to detect specific microorganisms on a surface. Initially, the amount of surface to be tested, the temperature of the environment, the amount of time to be allotted for detection, the types of modified bacteriophage and non-volatile media to be used and the like are all determined 300. A genetically modified bacteriophage is identified for the target microorganism 302. Next, the genetically modified parent bacteriophage, that will infect the target microorganism of interest, is combined with a non-volatile media for use on the surface and in the target surface environment, i.e., detection composition prepared 304. Enough detection composition is used to completely cover the surface of interest 306. Note that multiple strains of bacteriophage can be added to the media dependent on how many different microorganisms are of concern on the target surface. In some cases the different modified bacteriophage express the same detection marker, i.e., if any microorganism is present the same detection procedure is utilized. So for example, if either anthrax or Salmonella are present, detection marker is expressed. The user would not know which microorganism is present. However, bacteriophage can be modified to express different detection markers to provide additional information on which microorganism is present on the surface, for example, the modified bacteriophage for anthrax infection expresses luciferase and the bacteriophage for Salmonella infection expresses a fluorophore. Note that in all cases, the target microorganism would have to be alive in order to allow for bacteriophage infection and detection marker expression.

As shown in process 308, the temperature of the environment where the surface is located can be adjusted to maximize microorganism multiplication, thereby enhancing the bacteriophage infection and ultimately detection marker expression (note that in some instances the surface itself can be directly heated or cooled to accomplish the same enhanced amplification efficiency). Impregnated growth media, e.g., detection composition, is then applied to the target surface 310. Detection compositions of the present invention are sprayed or otherwise contacted to surfaces of concern. An even and consistent amount of detection composition is preferred on the surface so that each portion of the surface has a substantially consistent amount of phage and therefore should express a comparable level of detection marker. An appropriate amount of time is provided to allow for a detectable level or marker to be expressed by the phage if target microorganisms are present on the surface 312.

Note that positive and negative controls can be included in the process to facilitate the appropriate amount of time required for a detectable amount of marker to be expressed under the conditions of the target surface. In addition, positive and negative controls would provide assurances as to the quality of any given testing procedure. In one embodiment, a negative control includes an appropriate amount of detection composition and a sterile surface, the composition being layered onto the sterile surface at the same time the composition is applied to the target surface. The sterile surface provides an indication surface for the amount of background signal that results simply for having the modified phage on a surface. A positive control can also be included in the process where a surface having a known amount of microorganisms is tested for the same amount of time as the target surface. The positive control would provide an indication as to how much signal should be expressed if the known amount of microorganism is present.

In process 314, detection of the detection marker molecules would be marker molecule dependent, for example, detection of the fluorescent protein markers would be accomplished using the appropriate light source to cause fluorescence of the marker (Detection of a luciferase substrate could be detected by a luminometer, preferably a hand-held unit). Fluorescence would indicate the presence of the target microorganism. Interestingly, contact between genetically engineered phage and target microorganisms on a hard surface would ultimately result in the killing of the microorganism, thereby satisfying both the detection and thereby identification of the microorganisms presence but also the elimination of the microorganism. Detection results are compared too the positive and/or negative controls. In some embodiments, an additional microorganism killing step can be included, where the surface and the surfaces' environment would be disinfected to ensure that all microorganisms in the vicinity would be eliminated.

Embodiments of the invention provide three illustrative approaches: (1) a microorganism specific phage is genetically engineered to express a fluorophore, the phage being specific for a suspected microorganism contaminate on a surface; an agar media is prepared and impregnated with approximately 10⁵ engineered phage/ml; the agar media is sprayed on all of the surfaces of the surface; the temperature of the area housing the desk is elevated up to the optimum incubation temperatures for phage, to enhance phage amplification and expression; an allotted amount of time is allowed to pass, during which an appropriate light source is used to detect expression of the fluorophore. Presence of fluorophore indicates presence of the suspected microorganism on the surface, absence indicates that the desk did not contain viable target microorganism; (2) A similar approach as taken above, except that the media is a aqueous media having a high concentration of glycerol; and (3) the initial application media is water impregnated with the genetically engineered phage, followed by application of a polymer film onto the surfaces used to encapsulate the sprayed media.

The disclosed detection method offers a combination of specificity, sensitivity, simplicity, speed, and/or cost which is superior to any currently known microscopic organism detection method.

### Kits of the Invention:

The present invention also provides exemplary test kits for detecting a microorganism on a surface, as well as typical directions for using the test kit. A test kit of the invention preferably includes a container of bacteriophage buffer solution, a mixing container, one or more modified bacteriophage enclosed in a protective environment, appropriate growth media for the bacteriophage/target microorganism of interest and directions for using the kit. A receptacle for holding the foregoing test kit parts may also be provided. The kit embodiments of the invention also include a reference detection bacteriophage indicating the expected result if no bacteria are present (a negative control), i.e., the bacteriophage is engineered to express a detection marker in common with the bacteriophage of the kit, and have a small sterile surface to place it on within the same testing surface environment. This negative control would be run on a surface in the same environment as the target surface to provide an indication of background detection marker expression (where no microorganism is present). A positive control could also be provided in the kit, where a known amount of microorganism is placed on a kit surface that can be incubated in parallel with the surface testing conditions. A standard amount of positive control bacteriophage would be provided to indicate the detection marker signal expected when a given amount of microorganism is present.

The present invention may also provide an exemplary set of directions for using the test kits in accordance with the invention. Directions preferably comprise a sheet of paper with printed text and pictures illustrating the test procedure. The directions also illustrate an exemplary method for in situ detecting a microorganism.

Having generally described the invention, the same will be more readily understood by reference to the following examples, which are provided by way of illustration and are not intended as limiting.

1. A method for in situ detection of a target microorganism on a hard surface comprising:
preparing a detection composition wherein the detection composition comprises a non-volatile media impregnated with at least one bacteriophage having specificity for the target microorganism and wherein each of the at least one bacteriophage are modified to express a detectable marker when the target microorganism is present;
contacting the detection composition with the hard surface for a period of time sufficient to allow the modified bacteriophage to infect and express the detectable marker when the target microorganism is present and the target microorganism is alive; and
in situ detecting the presence of the detectable marker on the hard surface wherein presence of the detectable marker indicates presence of living target microorganism on the hard surface and absence of the detectable marker indicates absence of the target microorganism.

2. The method of claim 1 further comprising raising the temperature of an environment around the hard surface to facilitate target microorganism growth wherein enhanced target microorganism growth increases modified bacteriophage levels and detection marker production.

3. The method of claim 1 wherein the detectable marker is a fluorescent protein marker.

4. The method of claim 1 wherein the target microorganism is anthrax.

5. The method of claim 1 wherein the non-volatile media is a combination of glycerol and water.

6. The method of claim 1 wherein the non-volatile media is a combination of polyethylene glycol and water.

7. The method of claim 1 wherein the non-volatile media is a bacterial growth agar complementary for the target microorganism.

8. The method of claim 1 wherein the contacting of the detection composition and the hard surface is by spraying the detection composition onto the hard surface.

9. The method of claim 1 wherein the hard surface is located inside a building.

10. The method of claim 1 wherein the hard surface is located inside a vehicle.

11. A composition for in situ detection of a target microorganism on a hard surface comprising a non-volatile growth media impregnated with at least one genetically modified bacteriophage, the at least one genetically modified bacteriophage selective to the target microorganism wherein the genetically modified bacteriophage is capable of expressing a detectable marker if the bacteriophage contacts the target microorganism.

12. The composition of claim 11 wherein the non-volatile growth media is a combination of water and glycerol.

13. The compositions of claim 11 wherein the non-volatile growth media is a combination of water and polyethylene glycol.

14. The composition of claim 11 wherein the non-volatile growth media is a growth agar for the target microorganism.

15. The composition of claim 11 wherein the detectable marker is a fluorescent protein.

16. The composition of claim 11 wherein the target microorganism is anthrax.
